(19) United States
(12) Patent Application Publication
(10) Pub. No.: US 2010/0210835 A1
(43) Pub. Date: Aug. 19, 2010

(54) Mimetic Drug Delivery Systems for Release with Specific Molecular Triggers

(75) Inventor: Nicholas A. Peppas, Austin, TX (US)

Correspondence Address: CHALKER FLORES, LLP, 2711 LBJ FRWY, Suite 1036, DALLAS, TX, 75234, US

(73) Assignee: BOARD OF REGENTS, THE UNIVERSITY OF TEXAS SYSTEM, Austin, TX (US)

(21) Appl. No.: 12/708,224

(22) Filed: Feb. 18, 2010

Related U.S. Application Data
(60) Provisional application No. 61/153,605, filed on Feb. 18, 2009.

(51) Int. Cl. C07H 7/00; C07H 1/00

(52) U.S. Cl. 536/123; 536/123.1

(57) ABSTRACT

The present invention provides a molecule-imprinted reversible polymeric network composition and methods of making and using the same. In addition, the instant invention provides a composition for the delivery of an active agent. The composition includes a polymer matrix formed from at least a first and a second monomer and one or more active agents deposited within the polymer matrix. One or more recognition sites on at least one of the at least first and second monomers and an active site formed in the polymer matrix by the one or more recognition sites. The template binds to the active site by interacting with at least one of the one or more recognition sites, wherein the template causes the release of the one or more active agents.

### CROSS-REFERENCE TO RELATED APPLICATIONS

This application claims priority to U.S. Provisional Application Ser. No. 61/153,605, filed Feb. 18, 2009, the entire contents of which is incorporated herein by reference.

### TECHNICAL FIELD OF THE INVENTION

The present invention relates in general to the field of the controlled release of agents, and more particularly, to novel compositions and methods for making controlled release configurational biomimetic imprinting networks.

### BACKGROUND OF THE INVENTION

Without limiting the scope of the invention, its background is described in connection with making controlled release configurational biomimetic imprinting networks. Although the controlled delivery in pharmaceutical compositions has grown in the past decade, most classical controlled release systems provide only passive release. For example, the passive release may be due to a change in temperature or pressure resulting in a rupture and eventual release.

### SUMMARY OF THE INVENTION

The validity of polymer systems that display analyte-specific recognition has been established over the past several years. Opportunities utilizing this technology have been focused primarily on chromatography. With the work presented here, we show that molecular recognition may be expanded to combine recognition and release, reversible and non-reversible. Further work will show how these intelligent systems can be used for drug delivery, consumer products, biosensors and other applications.

The present invention provides a molecule-imprinted reversible polymeric network composition. The composition includes a polymer matrix formed from at least a first and a second monomer and one or more active agents deposited within the polymer matrix. One or more recognition sites on at least one of the at least first and second monomers and an active site formed in the polymer matrix by the one or more recognition sites. The template binds to the active site by interacting with at least one of the one or more recognition sites, wherein the template causes the release of the one or more active agents at a first concentration but not a second concentration.

The present invention provides a molecule-imprinted reversible polymeric network composition having a polymer matrix formed from at least a first and a second monomer, one or more active agents deposited within the polymer matrix, one or more recognition sites on at least one of the at least first and second monomers, an active site formed in the polymer matrix by the one or more recognition sites and a template reversibly bound to the active site by interacting with at least one of the one or more recognition sites, wherein the template causes the release of the one or more active agents at a first concentration but not a second concentration, wherein the first concentration or the second concentration is greater that the other concentration.

The present invention also provides a method of making a polymeric recognitive network by selecting one or more targets for recognition, selecting at lease a first and a second monomer to form a polymer matrix, wherein at least one of the at least first and second monomers comprise one or more recognition sites, assembling the polymer matrix to enclose one or more active agents, forming an active site in the polymer matrix wherein at least a portion of the one or more recognition sites are exposed to the template binds to the active site by interacting with at least one of the one or more recognition sites, wherein the template causes the release of the one or more active agents.

The present invention provides a method of selectively delivering an active agent by selecting one or more targets to initiate a selective delivery, selecting one or more active agents for selective delivery and forming a polymeric recognitive composition comprising a polymer matrix formed from at least a first and a second monomer, one or more active agents deposited within the polymer matrix, one or more recognition sites on at least one of the at least first and second monomers and an active site formed in the polymer matrix by the one or more recognition sites, wherein a template binds to the active site by interacting with at least one of the one or more recognition sites, wherein the template causes the release of the one or more active agents.

### BRIEF DESCRIPTION OF THE DRAWINGS

For a more complete understanding of the features and advantages of the present invention, reference is now made to the detailed description of the invention along with the accompanying figures and in which:

FIG. 1 is a schematic of the preparation of molecularly imprinted polymers.

FIG. 2 is a graph of the mass uptake and rupture of molecularly imprinted films due to presence of glucose.

FIG. 3 is a graph of the mass uptake and rupture of molecularly imprinted films due to presence of glucose or galactose.

FIG. 4 is a graph of the release of Nile Blue from MIP polymers in glucose solutions.

FIG. 5 is a graph of the release of Nile Blue from MIP polymers in water followed by 200 mg/dl glucose solution.

FIG. 6 is a graph of the release of Nile Blue from MIP Polymers in 200 mg/dl glucose solution followed by water.

FIG. 7 is a graph of the release of Nile Blue from MIP polymers in lactic acid solutions.

FIG. 8 is a graph of the release of Geraniol from MIP polymers in lactic acid solutions.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention provides a molecular imprinting delivery system that allows a respond to the presence of a specific molecule (or one or more molecules) in their environment and release their contents in response to that molecule and that molecule alone (or one or more molecules).

As used herein, the term “active ingredient(s),” “pharmaceutical ingredient(s),” “active agents” and “bioactive agent” are defined as drugs and/or pharmaceutically active ingredients. The present invention may be used to encapsulate, attach, bind or otherwise be used to affect the storage, stability, longevity and/or release of any of the following drugs as the pharmaceutically active agent in a composition. The active agent will generally be loaded according to the delivery needs at the location for release, e.g., at 0.01, 0.1, 0.5, 1.0, 2.0, 5.0, 10.0, 15.0, 20.0, 50.0, 100.0 or even 500.0 mg/ml of active agent by weight in solution prior to polymerization.

As used herein, the term “polymer matrix”, “polymeric matrix” or “molecularly imprinted polymer” refers to polymer a formed from at least a first and a second monomer (which may be the same monomer, e.g., a homopolymer) into which a recognition molecule is placed during the polymerization process and that is removed once the polymer is formed. In the present invention, the molecule that is recognized can include multiple recognition sites or components, such as a complex molecule or macromolecule. Examples of monomers that may be used include: poly(allylamine), acrylic acid, acrylamide, (diethylamino)ethyl methacrylate, (ethylamino)methacrylate, methacrylic acid, methylmethacrylate, triazacyclononane-copper(II) complex, 2-(methacryloyxloxy)ethyl phosphate, methacrylamide, 2-(trifluoromethyl)acrylic acid, 3-aminophenylboronic acid, poly(allylamine), o-phthalic dialdehyde, oleyl phenyl hydrogen phosphate, 4-vinylpyridine, vinylimidazole, 2-acryloilamido-2,2′-methopropane sulfonic acid, silica, organic silanes, N-(4-vinyl)-benzyl iminodiacetic acid, Ni(II)-nitrilotriacetic acid, N-acryloyl-alanine. These monomers may be combined with one or more crosslinkers to achieve the desired low or minimal swelling upon exposure, however, higher amounts of swelling also work with the present invention, as do higher concentrations of solvents. Examples of solvent include: ethylene glycol dimethacrylate, pentaerythritol triacrylate, pentaerythritol tetraacrylate, trimethylolpropane trimethacrylate, vinyl triethoxysilane, vinyl trimethoxysilane, toluene 2,4-diisocyanate, epichlorohydrin, triglycerolate diacrylate, polystyrene surface, Propylene glycol dimethacrylate, poly(ethylene glycol)N-dimethacrylate, methacrylate derived silica, acrylonitrile, N,N′-dimethylacrylamide, poly(ethylene glycol)diacrylate. Examples of solvents that may be used to achieve low or minimal swelling include acetonitrile, acetic acid, ethanol, aqueous buffer, toluene, water, chloroform, hexane, methanol, tetrahydrofuran.

As used herein, the term “recognition site” refers to a location within the polymer matrix around which a polymer is formed and that serves to recognize that a molecule or molecules are in the milieu in which the polymeric matrix is delivered and that causes the break-up of the polymeric matrix to release the active agent that is also incorporated into the polymeric matrix.

As used herein, the term “template” refers to a molecule that is used to create a site within the polymeric matrix into which a the same molecule can be reversibly bound the same way a molecule may fit into an the active site of an enzyme, which includes several parameters, e.g., shape, size, charges, in which a molecule use to “recognize” the site interacts and that causes the polymeric matrix or template to release of the one or more active agents, wherein the template causes the release of the one or more active agents at a first concentration but not a second concentration. In certain examples the first concentration is lower that the second concentration, or the second concentration is greater that the first concentration. In certain examples, the final composition may include one portion or layer that releases once a certain threshold concentration is reached but a separate portion does not (and there can be multiple layers or portions). The non-releasing portion(s) only release at a difference concentration of the recognized molecule, and so on, thereby providing a graduated response or release of the active agent based on the local milieu in which the final polymeric matrix is placed.

Non-limiting examples of active agents include, but are not limited to, antibiotics, analgesics, vaccines, anticonvulsants; antidiabetic agents, antifungal agents, antineoplastic agents, antiparkinsonian agents, antirheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. In certain embodiments, the one or more active agents are water-soluble, poorly water-soluble drug or a drug with a low, medium or high melting point. The active agents may be provided with or without a stabilizing salt or salts.

Active Agents

One or more of the following active agents may be combined with one or more carriers and the present invention (which may itself be the carrier):

Analgesic anti-inflammatory agents such as, acetaminophen, aspirin, salicylic acid, methyl salicylate, choline salicylate, glycol salicylate, 1-menthol, camphor, mefenamic acid, fluphenamic acid, indomethacin, diclofenac, alclofenac, ibuprofen, ketoprofen, naproxene, pranoprofen, fenoprofen, sulindac, fenbufen, clidanac, flurbiprofen, indoprofen, protizidic acid, fentiazac, tolmetin, tiaprofenic acid, bendazac, bufexamac, piroxicam, phenylbutazone, oxyphenbutazone, clofezone, pentazocine, mepirizole, and the like.

Drugs having an action on the central nervous system, for example sedatives, hypnotics, antianxiety agents, analgesics and anesthetics, such as, chloral, buprenorphine, naloxone, haloperidol, fluphenazine, pentobarbital, phenobarbital, secobarbital, amobarbital, cydobarbital, codeine, lidocaine, tetracaine, dyclonine, dibucaine, cocaine, procaine, mepivacaine, bupivacaine, etidocaine, prilocaine, benzocaine, fentanyl, nicotine, and the like.

Antihistaminics or antiallergic agents such as, diphenhydramine, dimenhydrinate, perphenazine, triprolidine, pyrilamine, chlorcyclizine, promethazine, carbinoxamine, tripelennamine, brompheniramine, hydroxyzine, cyclizine, meclizine, clorprenaline, terfenadine, chlorpheniramine, and the like. Anti-allergenics such as, antazoline, methapyrilene, chlorpheniramine, pyrilamine, pheniramine, and the like.

Decongestants such as phenylephrine, ephedrine, naphazoline, tetrahydrozoline, and the like.

Antipyretics such as aspirin, salicylamide, non-steroidal anti-inflammatory agents, and the like. Antimigrane agents such as, dihydroergotamine, pizotyline, and the like.

Acetonide anti-inflammatory agents, such as hydrocortisone, cortisone, dexamethasone, fluocinolone, triamcinolone, medrysone, prednisolone, flurandrenolide, prednisone, halcinonide, methylprednisolone, fludrocortisone, corticosterone, paramethasone, betamethasone, ibuprophen, naproxen, fenoprofen, fenbufen, flurbiprofen, indoprofen, ketoprofen, suprofen, indomethacin, piroxicam, aspirin, salicylic acid, diflunisal, methyl salicylate, phenylbutazone, sulindac, mefenamic acid, meclofenamate sodium, tolmetin, and the like.

Steroids such as, androgenic steriods, such as, testosterone, methyltestosterone, fluoxymesterone, estrogens such as, conjugated estrogens, esterified estrogens, estropipate, 17-β estradiol, 17-β estradiol valerate, equilin, mestranol, estrone, estriol, 17β ethinyl estradiol, diethylstilbestrol, progestational agents, such as, progesterone, 19-norprogesterone, norethindrone, norethindrone acetate, melengestrol, chlormadinone, ethisterone, medroxyprogesterone acetate, hydroxyprogesterone caproate, ethynodiol diacetate, norethynodrel, 17-α hydroxyprogesterone, dydrogesterone, dimethisterone, ethinylestrenol, norgestrel, demegestone, promegestone, megestrol acetate, and the like.

Respiratory agents such as, theophilline and β₂-adrenergic agonists, such as, albuterol, terbutaline, metaproterenol, ritodrine, carbuterol, fenoterol, quinterenol, rimiterol, solmefamol, soterenol, tetroquinol, and the like.

Sympathomimetics such as, dopamine, norepinephrine, phenylpropanolamine, phenylephrine, pseudoephedrine, amphetamine, propylhexedrine, arecoline, and the like.

Local anesthetics such as, benzocaine, procaine, dibucaine, lidocaine, and the like.

Antimicrobial agents including antibacterial agents, antifungal agents, antimycotic agents and antiviral agents; tetracyclines such as, oxytetracycline, penicillins, such as, ampicillin, cephalosporins such as, cefalotin, aminoglycosides, such as, kanamycin, macrolides such as, erythromycin, chloramphenicol, iodides, nitrofrantoin, nystatin, amphotericin, fradiomycin, sulfonamides, purrolnitrin, clotrimazole, miconazole chloramphenicol, sulfacetamide, sulfamethazine, sulfadiazine, sulfamerazine, sulfamethizole and sulfisoxazole; antivirals, including idoxuridine; clarithromycin; and other anti-infectives including nitrofurazone, and the like.

Antihypertensive agents such as, clonidine, α-methyldopa, reserpine, syrosingopine, rescinnamine, cinnarizine, hydrazine, prazosin, and the like. Antihypertensive diuretics such as, chlorothiazide, hydrochlorothrazide, bendoflumethazide, trichlormethiazide, furosemide, tripamide, methylclothiazide, penfluzide, hydrothiazide, spironolactone, metolazone, and the like. Cardiotonics such as, digitalis, ubidecarenone, dopamine, and the like. Coronary vasodilators such as, organic nitrates such as, nitroglycerine, isosorbitol dinitrate, erythritol tetranitrate, and pentaerythritol tetranitrate, dipyridamole, dilazep, trapidil, trimetazidine, and the like. Vasoconstrictors such as, dihydroergotamine, dihydroergotoxine, and the like. β-blockers or antiarrhythmic agents such as, timolol pindolol, propranolol, and the like. Humoral agents such as, the prostaglandins, natural and synthetic, for example PGE₁, PGE₂α, and PGF₂α, and the PGE₁ analog misoprostol. Antispasmodics such as, atropine, methantheline, papaverine, cinnamedrine, methscopolamine, and the like.

Calcium antagonists and other circulatory organ agents, such as, aptopril, diltiazem, nifedipine, nicardipine, verapamil, bencyclane, ifenprodil tartarate, molsidomine, clonidine, prazosin, and the like. Anti-convulsants such as, nitrazepam, meprobamate, phenytoin, and the like. Agents for dizziness such as, isoprenaline, betahistine, scopolamine, and the like. Tranquilizers such as, reserprine, chlorpromazine, and antianxiety benzodiazepines such as, alprazolam, chlordiazepoxide, clorazeptate, halazepam, oxazepam, prazepam, clonazepam, flurazepam, triazolam, lorazepam, diazepam, and the like.

Antipsychotics such as, phenothiazines including thiopropazate, chlorpromazine, triflupromazine, mesoridazine, piperracetazine, thioridazine, acetophenazine, fluphenazine, perphenazine, trifluoperazine, and other major tranqulizers such as, chlorprathixene, thiothixene, haloperidol, bromperidol, loxapine, and molindone, as well as, those agents used at lower doses in the treatment of nausea, vomiting, and the like.

Muscle relaxants such as, tolperisone, baclofen, dantrolene sodium, cyclobenzaprine.

Drugs for Parkinson's disease, spasticity, and acute muscle spasms such as levodopa, carbidopa, amantadine, apomorphine, bromocriptine, selegiline (deprenyl), trihexyphenidyl hydrochloride, benztropine mesylate, procyclidine hydrochloride, baclofen, diazepam, dantrolene, and the like. Respiratory agents such as, codeine, ephedrine, isoproterenol, dextromethorphan, orciprenaline, ipratropium bromide, cromglycic acid, and the like. Non-steroidal hormones or antihormones such as, corticotropin, oxytocin, vasopressin, salivary hormone, thyroid hormone, adrenal hormone, kallikrein, insulin, oxendolone, and the like.

Vitamins such as, vitamins A, B, C, D, E and K and derivatives thereof, calciferols, mecobalamin, and the like for dermatologically use. Enzymes such as, lysozyme, urokinaze, and the like. Herb medicines or crude extracts such as, Aloe vera, and the like.

Antitumor agents such as, 5-fluorouracil and derivatives thereof, krestin, picibanil, ancitabine, cytarabine, and the like. Anti-estrogen or anti-hormone agents such as, tamoxifen or human chorionic gonadotropin, and the like. Miotics such as pilocarpine, and the like.

Cholinergic agonists such as, choline, acetylcholine, methacholine, carbachol, bethanechol, pilocarpine, muscarine, arecoline, and the like. Antimuscarinic or muscarinic cholinergic blocking agents such as, atropine, scopolamine, homatropine, methscopolamine, homatropine methylbromide, methantheline, cyclopentolate, tropicamide, propantheline, anisotropine, dicyclomine, eucatropine, and the like.

Mydriatics such as, atropine, cyclopentolate, homatropine, scopolamine, tropicamide, eucatropine, hydroxyamphetamine, and the like. Psychic energizers such as 3-(2-aminopropy)indole, 3-(2-aminobutyl)indole, and the like.

Antidepressant drugs such as, isocarboxazid, phenelzine, tranylcypromine, imipramine, amitriptyline, trimipramine, doxepin, desipramine, nortriptyline, protriptyline, amoxapine, maprotiline, trazodone, and the like.

Anti-diabetics such as, insulin, and anticancer drugs such as, tamoxifen, methotrexate, and the like.

Anorectic drugs such as, dextroamphetamine, methamphetamine, phenylpropanolamine, fenfluramine, diethylpropion, mazindol, phentermine, and the like.

Anti-malarials such as, the 4-aminoquinolines, alpha-aminoquinolines, chloroquine, pyrimethamine, and the like.

Anti-ulcerative agents such as, misoprostol, omeprazole, enprostil, and the like.

Antiulcer agents such as, allantoin, aldioxa, alcloxa, N-methylscopolamine methylsuflate, and the like. Antidiabetics such as insulin, and the like.

For use with vaccines, one or more antigens, such as, natural, heat-killer, inactivated, synthetic, peptides and even T cell epitopes (e.g., GADE, DAGE, MAGE, etc.) and the like.

The drugs mentioned above may be used in combination as required. Moreover, the above drugs may be used either in the free form or, if capable of forming salts, in the form of a salt with a suitable acid or base. If the drugs have a carboxyl group, their esters may be employed.

The acid mentioned above may be an organic acid, for example, methanesulfonic acid, lactic acid, tartaric acid, fumaric acid, maleic acid, acetic acid, or an inorganic acid, for example, hydrochloric acid, hydrobromic acid, phosphoric acid or sulfuric acid. The base may be an organic base, for example, ammonia, triethylamine, or an inorganic base, for example, sodium hydroxide or potassium hydroxide. The esters mentioned above may be alkyl esters, aryl esters, aralkyl esters, and the like.

When a drug different than an anesthetic agent is used the solvent selected is one in that the drug is soluble. In generally the polyhydric alcohol may be used as a solvent for a wide variety of drugs. Other useful solvents are those known to solubilize the drugs in question.

Bioactive Delivery.

The bioactive may also be administered parenterally, intraperitoneally, intraspinally, or intracerebrally. Dispersions may be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use sterile powders for the preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin.

The composition may be orally administered, for example, with an inert diluent or an assimilable edible carrier. Other ingredients may also be enclosed in a hard or soft shell gelatin capsule, compressed into tablets, or incorporated directly into the subject's diet. For oral therapeutic administration, the active agent may be incorporated with excipients and used in the form of ingestible tablets, buccal tablets, troches, capsules, elixirs, suspensions, syrups, wafers, and the like. The percentage of the active agent in the compositions and preparations may, of course, be varied. The amount of the active agent in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of active agent calculated to produce the desired therapeutic effect in association with the required pharmaceutical carrier. The specification for the dosage unit forms of the invention are dictated by and directly dependent on (a) the unique characteristics of the active agent and the particular therapeutic effect to be achieved, and (b) the limitations inherent in the art of compounding such an active agent for the treatment of a selected condition in a subject.

Aqueous compositions of the present invention comprise an effective amount of the nanoparticle, nanofibril or nanoshell or chemical composition of the present invention dissolved and/or dispersed in a pharmaceutically acceptable carrier and/or aqueous medium.

The biological material should be extensively dialyzed to remove undesired small molecular weight molecules and/or lyophilized for more ready formulation into a desired vehicle, where appropriate. The active compounds may generally be formulated for parenteral administration, e.g., formulated for injection via the intravenous, intramuscular, sub-cutaneous, intralesional, and/or even intraperitoneal routes. The preparation of an aqueous compositions that contain an effective amount of the nanoshell composition as an active component and/or ingredient will be known to those of skill in the art in light of the present disclosure. Typically, such compositions may be prepared as injectables, either as liquid solutions and/or suspensions; solid forms suitable for using to prepare solutions and/or suspensions upon the addition of a liquid prior to injection may also be prepared; and/or the preparations may also be emulsified.

Dosage Forms.

The examples of pharmaceutical preparations described above are merely illustrative and not exhaustive; the nanoparticles of the present invention are amenable to most common pharmaceutical preparations.

As used herein the term “monomer” as used in the context of first and second monomers refers to molecules that are polymerizable, that is, can be polymerized in situ using, e.g., cross-linking agents, catalysts, are self-polymerizing or are polymerizable using heat, UV-light, pH or other external sources of polymerization. Examples of monomers that can be used with the present invention are those that are generally biocompatible and/or biodegradable and that for polumeys with units that are polyanhydrides, polyorthoesters, polyhydroxy acids, polydioxanones, polycarbonates, and polyaminocarbonates. Non-limiting examples of suitable polymers for use with the present invetion include synthetic polymers such as poly(ethylene glycol), poly(ethylene oxide), partially or fully hydrolyzed poly(vinyl alcohol), poly(vinylpyrrolidone), poly(ethyloxazoline), poly(ethylene oxide)-co-poly(propylene oxide) block copolymers (poloxamers and meroxapols), poloxamines, carboxymethyl cellulose (and derivatives), and hydroxyalkylated celluloses (and derivatives) such as hydroxyethyl cellulose and methylhydroxypropyl cellulose, and natural polymers such as polypeptides, polysaccharides or carbohydrates such as Ficoll®, polysucrose, hyaluronic acid, dextran (and derivatives), heparan sulfate, chondroitin sulfate, heparin, or alginate, and proteins such as gelatin, collagen, albumin, or ovalbumin or copolymers or blends thereof. Generally, the monomer and/or oligomer may include a biodegradable linkage such as amide-, anhydride-, carbonate-, ester-, or orthoester linkages; more preferably, an anhydride-linkage so that the polymer network formed by the monomer and/or oligomer is biodegradable. In certain examples, the polymer will be formed using an initiator, e.g., a photoinitiator, a redox initiator, or a combination thereof.

Recognition in nature is a complex orchestration of numerous interactions between individual atoms and cumulative interactions between secondary structures. For example, the active sites of enzymes are composed of several amino acid residues, which covalently bind ligand molecules in a very specific manner. However, the activity of the site is dependent on the stabilization of the three-dimensional structure by the interactions of hundreds of other residues within the structure of secondary and tertiary domains. The term configurational biomimesis refers to the three-dimensional arrangement of chemical groups that can specifically bind a biomolecule via noncovalent forces. This designed recognition involves analyzing the molecular basis of recognition in biological systems and attempts to mimic similar interactions on a molecular level. Molecularly imprinted polymers (MIPs) are polymers that are formed in the presence of an imprinted compound or targeting chemical, biological or other molecule such that the imprinted compound may later be removed, leaving a MIP that is able to recognize and bind to the imprinted compound via a binding cavity, perhaps even able to differentiate with isomeric specificity.¹⁻⁵

The present invention has a large number of potential applications given its design of a precise macromolecular chemical architecture that can recognize target molecules from an ensemble of closely related molecules. The main thrust in this field includes separation processes (e.g., chromatography, capillary electrophoresis, solid-phase extraction, membrane separations), immunoassays and antibody mimics, biosensor recognition elements, and catalysis and artificial enzymes. However, relatively little attention has been paid to controlled delivery.⁶⁻¹¹

The present invention provides a molecule-imprinted reversible polymeric network composition. The composition includes a polymer matrix formed from at least a first and a second monomer and one or more active agents deposited within the polymer matrix. One or more recognition sites on at least one of the at least first and second monomers and an active site formed in the polymer matrix by the one or more recognition sites. The template binds to the active site by interacting with at least one of the one or more recognition sites, wherein the template causes the release of the one or more active agents.

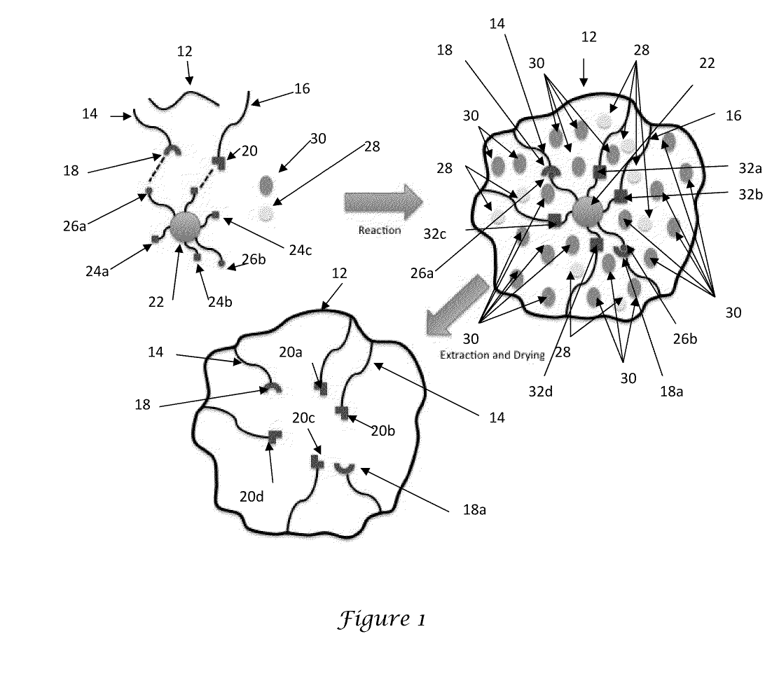

Configurational biomimesis and nanoimprinting create stereo-specific three-dimensional binding cavities based on the template of interest. Configurational biomimetic imprinting techniques of the present invention involve forming a pre-polymerization complex between the template molecule and functional monomers or functional oligomers (or polymers) with specific chemical structures designed to interact with the template either by covalent, non-covalent chemistry (self-assembly) or both, as seen in FIG. 1.

FIG. 1 is a schematic of the preparation of molecularly imprinted polymers. Monomers 12, 14 and 16 may be connected together to leave a first recognition site 18 on monomer 14 and a second recognition site 20 on monomer 16 accessible. The template 22 includes a first complimentary recognition site 26a that recognizes first recognition site 18 on monomer 14. In some instances, there are multiple first complimentary recognition sites 26b that recognizes first recognition site 18 on monomer 14. The template 22 includes a second complimentary recognition sites 24a-24c that recognizes second recognition site 20 on monomer 16. The monomers 12, 14 and 16 interconnect and the template aligns the first recognition site 18 and the first complimentary recognition site 26a and the second complimentary recognition sites 24a-24c that recognizes second recognition site 20. The interconnection positions solvents 28 and initiators 30 within or between the monomers 12, 14 and 16 and the template 22.

When the molecularly imprinted polymer of the instant invention is formed the template 22 positions the first complimentary recognition site 26a and the first recognition site 18 on monomer 14 and the second complimentary recognition sites 24a-24c that recognizes second recognition site 20 on monomers 16. The monomer 12 can then connect monomers 14 and 16. Monomers 12, 14 and 16 are connected and the first recognition site 18 on monomer 14 and second recognition site 20 on monomer 16 bind to the template 22. The first complimentary recognition site 26a that recognizes first recognition site 18 on monomer 14 (or in some instances, there are multiple first complimentary recognition sites 26b that recognizes first recognition site 18 on monomer 14) and the second complimentary recognition sites 24a-24c binds the second recognition site 20 to form complex 32a-32d on monomers 16. The monomers 12, 14 and 16 interconnect and the template aligns the first recognition site 18 and the first complimentary recognition site 26a and the second complimentary recognition sites 24a-24c that recognizes second recognition site 20. The interconnection positions solvents 28 and initiators 30 within or between the monomers 12, 14 and 16 and the template 22.

The template 22 may be removed from the molecularly imprinted polymer to leave only the monomers 12, 14 and 16 connected together to leave the first recognition site 18 on monomer 14 and a second recognition site 20 on monomer 16 accessible. The molecularly imprinted polymer retains the structure but lacks the template 22.

Proper tuning of non-covalent interactions such as increasing macromolecular chain hydrophobicity, including strong ionic directed recognition sites with hydrophobic domains, or including stronger hydrogen bond donors and acceptors, has been shown to enhance binding and achieve selective recognition in aqueous solutions. Thermodynamic analysis regarding energy contributions of ligand-receptor binding outlines the importance of directed tuning of these parameters in non-covalent recognition.

The present invention provides polymer systems that display analyte-specific recognition that may be expanded to combine recognition and release, reversible and non-reversible. The instant invention provides intelligent systems for drug delivery, consumer products, biosensors and other applications.

The present invention (also called the AFFINIMER™ system) is a molecularly imprinted polymer coupled with an active agent that responds in a unique way to the presence of the analyte to which it has been imprinted. It does not simply bind and sequester the analyte, but the polymer itself swells and can be made to rupture due to the presence of the analyte. This creates a system that not only recognizes, but recognizes and releases.

This system gives the flexibility to provide release upon not one, but many different possible triggers. For example, one of these intelligently designed systems is currently being used to selectively recognize and respond to variations in analyte concentrations and trigger a controlled, dose appropriate, level of active determined by this recognition event. Therefore, one of the primary advantage of the present invention is that the components can be evaluated against numerous analytes and environmental triggers to determine which combination of analyte components will have the most desirable protection and release characteristics. This range of triggering opportunities allows utilization of this technology to enhance release performance in a number of products and applications.

Utilization of environmentally relevant analytes, biomarkers and conditions (i.e. the use of a specific molecule or environmental trigger) allow for selective activated release only at the desired point of use or application. Furthermore, this approach, coupled with the use of a robust intelligent material, provides the necessary level of protection of active during storage and controlled rate of release upon activation.

These systems allow reversible or non-reversible release, depending on the design of the system, of incorporated active agents. The release will happen selectively due to the presence of the imprinted active agent and can be made to be proportional to the amount of agent present or can be an “all or nothing” release when the concentration of the active agent reaches a desired or critical amount. Again, these different formulation types can be prepared using the same basic imprinted polymer structure, then utilizing a variety of controlled release designs and techniques to give reversible, non-reversible, proportional or complete release of the desired agent. The results that will be presented in this paper confirmed the sensitivity and selectivity of the triggering mechanism as well as quantitative and qualitative release profiles with dose response curves.

The films of the instant invention are based on, e.g., copolymers of methacrylic acid (MAA) crosslinked with triethyleneglycol dimethacrylate (TEGDMA). A number of different monomers as well as crosslinking agents and the combination described here have proven to yield the best balance of imprinted sensitivity, selectivity and physical strength. Although many research groups use ethyleneglycol dimethacrylate (EGDMA) for the crosslinking agent for MIP systems, the instant invention found TEGDMA crosslinked films to have a much better physical integrity. The concentrations of glucose and lactic acid used for exposure of the films desribed herein to evaluate their mimetic nature are concentrations that are physiologically relevant. The high concentration of 200 μg/dl is, oddly enough, a high concentration for both glucose in the blood and lactic acid in human sweat.

Nile Blue was chosen as an active surrogate for its hydrophobic nature (similar to essential oils, fragrances, and Vitamin E) and the ability to capture visible results in the laboratory. Geraniol is a potential active agent a number of applications. More hydrophilic agents have been studied but are not presented here because of space limitations.

Preparation of Films. Water (4 ml) and ethanol (4.5 ml) were pipetted into a 25 ml glass container. Lactic acid (60 mg) was added and the mixture sonicated for 5 minutes, followed by the addition of 0.39 g of methacrylic acid. The mixture was again sonicated for five minutes and left at room temperature for a further five minutes. TEGDMA (3.1 g) was added and the mixture vortexed. Initiator (DMPA, 50 mg) was added and the mixture degassed with nitrogen for 3 minutes.

Thin films were prepared in molds consisting of glass slides with appropriate spacers. Once the polymer solution was degassed, the molds were filled with the solution using a 100 ml pipettor, making a note of volume introduced. This was done quickly and with minimum light exposure to avoid undesired early polymerization. Once filled, the molds were immediately placed under the UV lamp. The lamp position was calibrated to irradiate the slides at 15,000 mwatts/cm². The films were irradiated for five minutes at which time they were opaque and solid.

The slides were carefully separated and the slide containing the film was washed gently in a beaker containing 10% methanol/water. Once washed, the films were rinsed with distilled water, placed in plastic containers and covered with water. Films were washed at room temperature for five days with the water being changed twice daily. At the end of the five days the films were carefully removed and allowed to dry. Once dry, 7 mm discs were carefully cut out using a standard stainless steel cork borer.

Loading of Films with Nile Blue For Release Studies. For dye loading, 50 mg of Nile Blue was weighed out and dissolved in 100 ml of distilled water, resulting in a final concentration of 0.5 mg/ml. The mixture was vigorously mixed and sonicated for 5 minutes. In the meantime discs were arranged in a segmented plastic container and the Nile Blue solution gently pipetted onto the discs until they were fully covered. The container was covered and left at room temperature overnight.

The following day the Nile Blue was gently removed by pipettes until the discs were visible. The discs were carefully removed and washed three times with water and then placed in a clean container and covered in water. The container was then placed on a hot plate set to 55° C. and left for three hours. The water was removed and the process repeated. At the end of six hours the discs were removed and placed on a clean foil and dried overnight at room temperature. The following day the discs were ready for study.

Loading of films with and Active Agent. The active agent geraniol was used for release studies. Once dry, 7 mm discs were carefully cut out using a standard stainless steel cork borer. The discs were lifted out and placed on a clean dish. For the loading, 100 mg of geraniol was weighed out and dissolved in 100 ml of 50% ethanol:distilled water, resulting in a final concentration of 1.0 mg/ml.

The mixture was vigorously mixed and sonicated for 5 minutes. In the meantime discs were arranged in a segmented plastic container and the geraniol solution gently pipetted onto the discs until they were fully covered. The container was tightly covered and left at room temperature overnight. The following day the geraniol solution was gently removed by pipette until the discs were visible. The discs were washed three times with water and placed on a clean foil and left to dry overnight.

Preparation of lactic acid solutions. Lactic acid (200 mg) was weighed out and dissolved in 100 ml of water, resulting in a final concentration of 200 mg/dl. Serial dilutions in water resulted in concentrations of 100 mg/dl, 50 mg/dl, 25 mg/dl, 12.5 mg/dl and 0 mg/ml. The pH of all lactic acid dilutions was adjusted to a pH of 5.0 by the addition of sodium hydroxide

Preparation of glucose solutions. Glucose (200 mg) was weighed out and dissolved in 100 ml of water, resulting in a final concentration of 200 mg/dl. Serial dilutions in water resulted in concentrations of 100 mg/dl, 50 mg/dl and 0 mg/ml.

Incubation—Nile Blue. The above solutions of glucose or lactic acid were used as triggers for Nile Blue release. 4 ml of each solution was placed into three petri dishes kept at room temperature. Stained discs were placed into each dish and the dish covered. Samples were taken (400 ul) into micro-cuvettes at specific time points and the concentration of Nile Blue present was determined by measuring the absorbance of the Nile Blue solutions at 625 nm.

Incubation Study—Geraniol. The above solutions of glucose were used as triggers to assess the concentration profile of geraniol release. 4 ml of each solution was placed into three petri dishes kept at room temperature. Stained discs were placed into each dish and the dish covered. The stop clock started and samples were taken (100 ul) into HPLC autosampler vials and the concentration of geraniol present was determined by injecting onto a C18 HPLC column and monitoring at a wavelength of 195 nm.

Assay for Geraniol. Geraniol concentration was quantified using an HPLC Diode array detector (DAD) at 195 nm. After blanking with water, samples were injected and the peak area of the geraniol peak recorded. Intermixed with the samples were standards of geraniol of known concentration made from the staining solution. This allowed for the construction of a standard curve to determine the concentration of geraniol in the incubation samples.

Film Swelling and Rupture as Evidence of Response. The mimetic polymer films have shown to rupture due to the presence of the trigger molecule for which they were prepared. These studies evaluated the swelling of the polymer films in desired solutions of glucose at various concentrations as well as in solutions of extremely similar molecules, such as galactose which differs from glucose in the —OH and —H bonding at only one carbon atom.

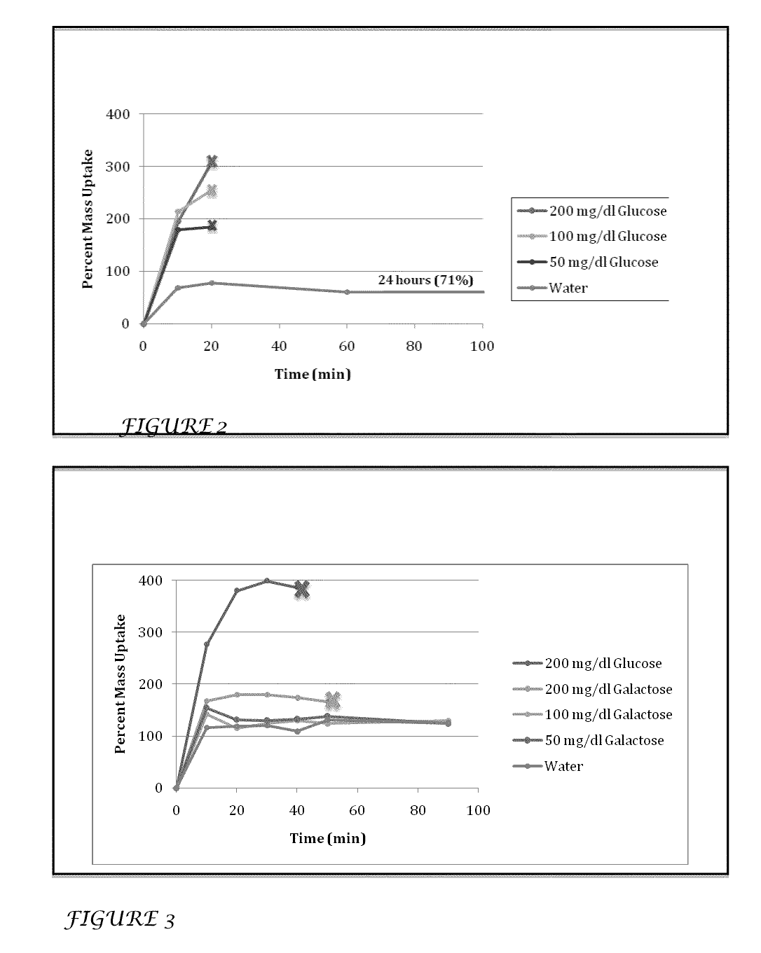

FIG. 2 is a graph of the mass uptake and rupture of molecularly imprinted films due to presence of glucose. In FIG. 2, the rupture of films is denoted by “X”. As seen in FIG. 2, that the MIP films of the instant invention swelled at progressively higher levels when in higher concentrations of glucose. Rupture occurred at the earliest times for films in concentrations of 200 μg/dl glucose (e.g., high but possible physiological level) with no rupture being seen in water even after 24 hours. Studies were repeated in the same concentrations of galactose as showed rupture with the glucose solutions and only the 200 mg/dl galactose solution caused rupture of the polymer film and with a much lower degree of swelling than the film showed in a similar glucose solution. The swelling in all solutions of galactose at less than 200 mg/dl showed swelling indistinguishable from that in water with no rupture as shown in FIG. 3. FIG. 3 is a graph of the mass uptake and rupture of molecularly imprinted films due to presence of glucose or galactose. In FIG. 3, the rupture of films is denoted by “X”.

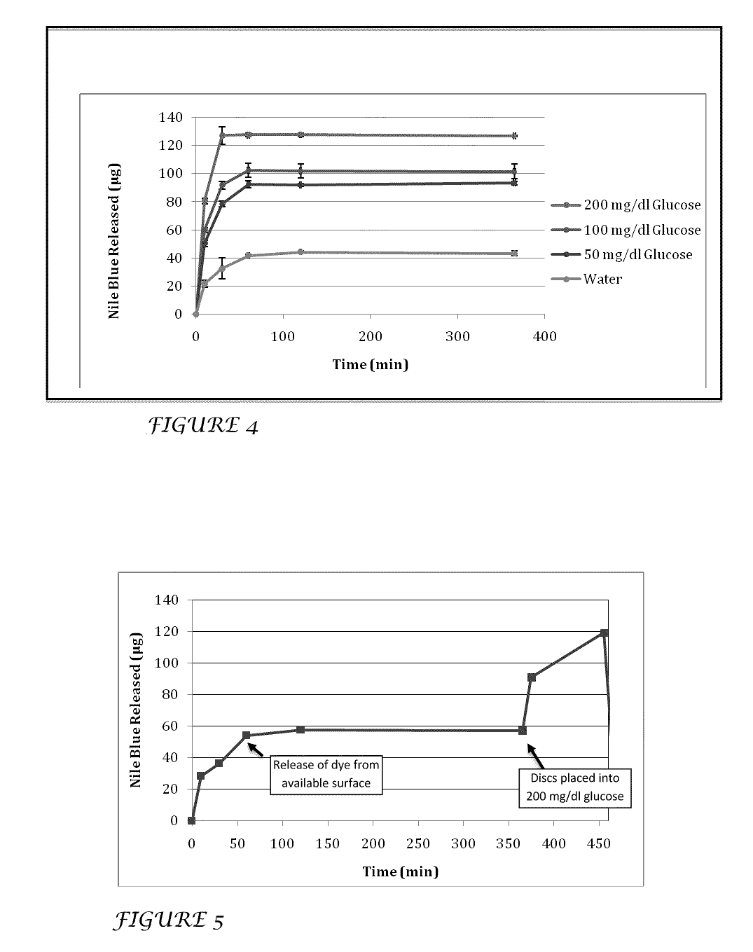

Although the swelling tests showed the structural changes that result when the mimetic polymer films are exposed to their target analyte, the true test of performance would be release of an unrelated compound from those films. This performance was tested with release of Nile Blue as described previously. FIG. 4 is a graph of the release of Nile Blue from MIP polymers in glucose solutions. FIG. 4 shows some release data where there is still some release into the water as the washing steps had not yet been optimized. Even with some release of Nile Blue in the water, it is clear that the Nile Blue is released in increasing amounts as the concentration of glucose in the solution increases.

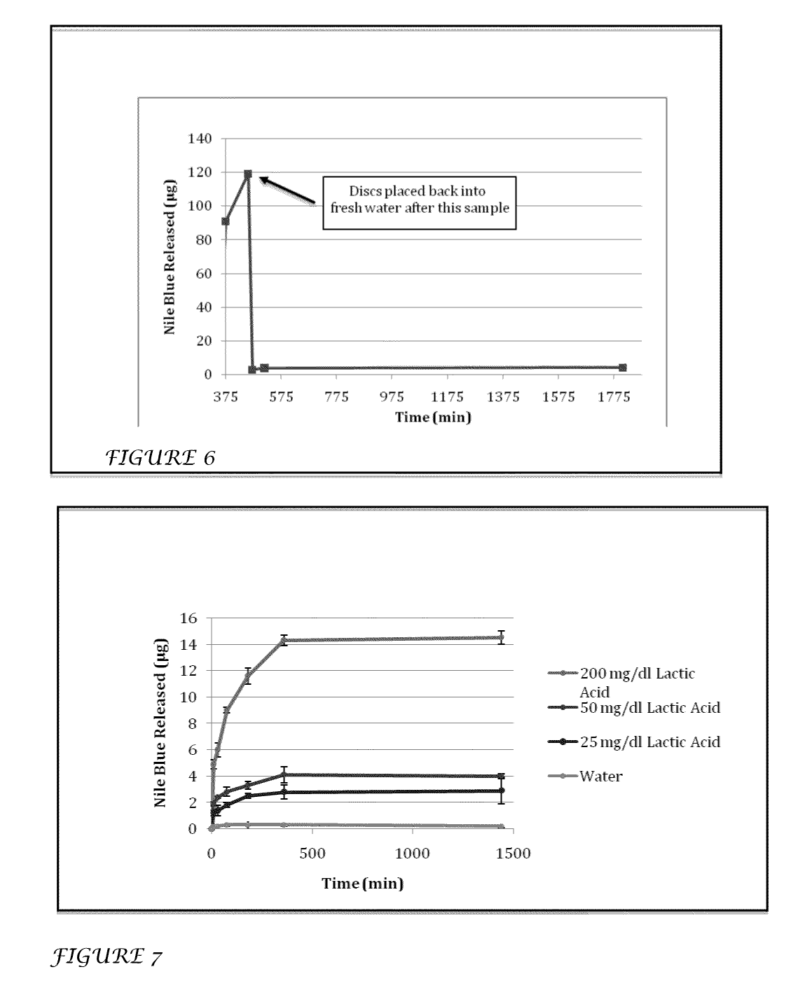

FIG. 5 is a graph of the release of Nile Blue from MIP polymers in water followed by 200 mg/dl glucose solution. The films that had been in water, and that had not showed any significant release for about 5 hours, at 365 minutes were removed from the water and placed in a solution of 200 mg/dl glucose. The release of Nile Blue from these films began almost immediately even though their release in water alone had stopped as shown in FIG. 5. To evaluate the true reversibility of these systems, at 450 minutes the films were removed from the glucose solution and placed in fresh water. The release stopped almost immediately as shown in FIG. 6. FIG. 6 is a graph of the release of Nile Blue from MIP Polymers in 200 mg/dl glucose solution followed by water.

FIG. 7 is a graph of the release of Nile Blue from MIP polymers in lactic acid solutions. Imprinted films have also been prepared that are sensitive to the presence of lactic acid and release of Nile Blue from those films is shown in FIG. 7.

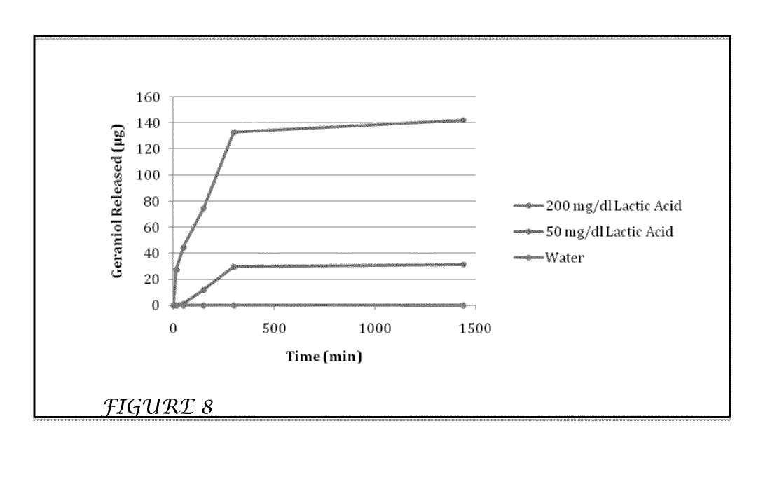

Geraniol was loaded into MIP films prepared that are triggered either by lactic acid. The loading in these films was approximately 145 μg per disc. FIG. 8 is a graph of the release of Geraniol from MIP polymers in lactic acid solutions. FIG. 8 shows the release of geraniol as triggered by lactic acid, with nearly 100% release of geraniol at 200 mg/dl of lactic acid at 24 hours.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Bergmann N M, Peppas N A. Molecularly imprinted polymers with specific recognition for macromolecules and proteins, Prog. Polym. Sci, 2008; 33(3), 271-288.

2. Schillemans J P, van Nostrum C F. Molecularly imprinted polymer particles: synthetic receptors for future medicine, Nanomedicine, 2006, 437-447

3. Silvestri D, Barbani N, Cristallini C, Giusti P, Ciardelli G. Molecularly imprinted membranes for an improved recognition of biomolecules in aqueous medium, J Membrane Sci., 2006; 282, 284-295.

4. Bossi A, Bonini F, Turner A P F, Piletsky S A. Molecularly imprinted polymers for the recognition of proteins: The state of the art, Biosen. Bioelectr., 2007; 22, 1131-1137.

5. Wei S, Molinelli A, Mizaikoff B. Molecularly imprinted micro and nanospheres for the selective recognition of 17β-estadiol, Biosen. Bioelectr., 2006; 21, 1943-1951.

6. Hilt J Z, Byrne M E. Configurational biomimesis in drug delivery: molecular imprinting of biologically significant molecules, Adv. Drug Del. Rev, 2004; 26, 1599-1620.

7. Duarte A R C, Casimiro T, Aguiar-Ricardo A, Simplicio A L, Duarte C M M. Supercritical fluid polymerization and impregnation of molecularly imprinted polymers for drug delivery, J. Supercit. Fluids, 2006; 39, 102-106.

8. Bures P, Huang Y, Oral E, Peppas N A. Surface modifications and molecular imprinting of polymers in medical and pharmaceutical applications, J Contr. Rel, 2001; 75, 25-33.

9. Alvarez-Loranzo C, Concheiro A. Molecularly imprinted polymers for drug delivery, J Chromatography B, 2004; 804, 231-245.

10. Sellergren B, Allender C J. Molecularly imprinted polymers: A bridge to advanced drug delivery, Adv. Drug Del. Rev., 2005; 57, 1733-1741.

11. Ciardelli G, Cioni B, Cristallini C, Barbani N, Silvestri D, Giusti P. Acrylic polymeric nanospheres for the release and recognition of molecules of clinical interest, Biosen. Bioelectr., 2004; 20, 1083-1090.

What is claimed is:

1. A molecule-imprinted reversible polymeric network composition comprising:
a polymer matrix formed from at least a first and a second monomer;
one or more active agents deposited within the polymer matrix;
one or more recognition sites on at least one of the at least first and second monomers; and
an active site formed in the polymer matrix by the one or more recognition sites, wherein a template binds to the active site by interacting with at least one of the one or more recognition sites, wherein the template causes the release of the one or more active agents at a first concentration but not a second concentration.

2. The composition of claim 1, wherein the template comprises an imprinted compound, targeting chemical, biological molecule, organic molecule, macromolecule, carbohydrates, lipids, proteins and nucleic acids.

3. The composition of claim 1, wherein the template binds reversibly.

4. The composition of claim 1, wherein the at least a first and a second monomer comprise methacrylic acid (MAA), triethyleneglycol dimethacrylate (TEGDMA), ethyleneglycol dimethacrylate (EGDMA) or combinations thereof.

5. The composition of claim 1, wherein the polymer matrix is adapted for pharmaceuticals, medical agents, food components, detergents, bleaches, fabric softeners, fragrances, cosmetic products, air fresheners, room deodorant devices, perfumed substrates, perfumed plastics and pet collars.

6. The composition of claim 1, wherein the template is a pharmaceutical, medical agents, food components, detergents, bleaches, fabric softeners, fragrances, cosmetic products, air fresheners, room deodorant devices, perfumed substrates, perfumed plastics and pet collars.

7. The composition of claim 1, wherein the template comprises a carbohydrate polymer of glycosidic type mono-sugar repeative units, galactomannans, pectins, alginates, carrageenans and xanthan gum that are linear or branched, neutral or anionic and combinations thereof.

8. The composition of claim 1, wherein the active agent is released upon a change in solubility, pressure, a pH shift, a change in temperature, a temperature increase, enzymatic breakdown, diffusion and combinations thereof.

9. The composition of claim 1, wherein the polymer matrix is formed into one or more layers.

10. The composition of claim 1, wherein the polymer matrix is formed into one or more layers, each of which recognizes one or more different molecules and each of which provides a barrier to the release of one or more different active or inert agents or both.

11. The composition of claim 1, wherein the polymer matrix is formed into a sphere, film, planar, semi-spherical, cylinder, rod, hemispheres, conical, hemi-cylinders and combination thereof

12. A molecule-imprinted reversible polymeric network composition comprising:
a polymer matrix formed from at least a first and a second monomer;
one or more active agents deposited within the polymer matrix;
one or more recognition sites on at least one of the at least first and second monomers;
an active site formed in the polymer matrix by the one or more recognition sites; and
a template reversibly bound to the active site by interacting with at least one of the one or more recognition sites, wherein the template causes the release of the one or more active agents.

13. A method of making a polymeric recognitive network comprising the steps of:
selecting one or more targets for recognition;
selecting at lease a first and a second monomer to form a polymer matrix, wherein at least one of the at least first and second monomers comprise one or more recognition sites;
assembling the polymer matrix to enclose one or more active agents; and
forming an active site in the polymer matrix wherein at least a portion of the one or more recognition sites are exposed to the template binds to the active site by interacting with at least one of the one or more recognition sites, wherein the template causes the release of the one or more active agents.

14. A method of selectively delivering an active agent comprising the steps of:
selecting one or more targets to initiate a selective delivery;
selecting one or more active agents for selective delivery; and
forming a polymeric recognitive composition comprising a polymer matrix formed from at least a first and a second monomer, one or more active agents deposited within the polymer matrix, one or more recognition sites on at least one of the at least first and second monomers and an active site formed in the polymer matrix by the one or more recognition sites, wherein a template binds to the active site by interacting with at least one of the one or more recognition sites, wherein the template causes the release of the one or more active agents.

15. The method of claim 14, wherein the selective delivery comprises an extended release, immediate release, delayed release, stepped release, bimodal or combination thereof.

16. The method of claim 14, wherein the template comprises an imprinted compound, targeting chemical, biological molecule, organic molecule, macromolecule, carbohydrates, lipids, proteins, a carbohydrate polymer of glycosidic type mono-sugar repeative units, galactomannans, pectins, alginates, carrageenans and xanthan gum that are linear or branched, neutral or anionic and nucleic acids.

17. The method of claim 14, wherein the template binds reversibly.

18. The method of claim 14, wherein the at least a first and a second monomer comprise methacrylic acid (MAA), triethyleneglycol dimethacrylate (TEGDMA), ethyleneglycol dimethacrylate (EGDMA) or combinations thereof.

19. The method of claim 14, wherein the polymer matrix is adapted for pharmaceuticals, medical agents, food components, detergents, bleaches, fabric softeners, fragrances, cosmetic products, air fresheners, room deodorant devices, perfumed substrates, perfumed plastics and pet collars, a pharmaceutical, medical agents, food components, detergents, bleaches, fabric softeners, fragrances, cosmetic products, air fresheners, room deodorant devices, perfumed substrates, perfumed plastics and pet collars.

20. The method of claim 14, wherein the active agent is released upon a change in solubility, pressure, a pH shift, a change in temperature, a temperature increase, enzymatic breakdown, diffusion and combinations thereof.

21. The method of claim 14, wherein the polymer matrix is formed into one or more layers.

22. The method of claim 14, wherein the polymer matrix is formed into one or more layers, each of which recognizes one or more different molecules and each of which provides a barrier to the release of one or more different active or inert agents or both.

23. The method of claim 14, wherein the polymer matrix is formed into a sphere, film, planar, semi-spherical, cylinder, rod, hemispheres, conical, hemi-cylinders and combination thereof.

24. A recognitive polymeric matrix made by a method comprising:
selecting one or more targets for recognition;
selecting at lease a first and a second monomer to form a polymer matrix, wherein at least one of the at least first and second monomers comprise one or more recognition sites;
assembling the polymer matrix to enclose one or more active agents; and
forming an active site in the polymer matrix wherein at least a portion of the one or more recognition sites are exposed to the template binds to the active site by interacting with at least one of the one or more recognition sites, wherein the template causes the release of the one or more active agents in an amount that correlates with the amount of the target in solution, wherein an increase in the target in solution causes an increase in the release of the active agent.
